(19) United States
(12) Patent Application Publication
(10) Pub. No.: US 2006/0239925 A1
(43) Pub. Date: Oct. 26, 2006

(54) Method of manufacturing pharmaceutical preparation containing liposomes

(75) Inventors: Takeshi Wada, Tokyo (JP); Eiichi Ueda, Tokyo (JP); Yasuyuki Motokui, Tokyo (JP)

Correspondence Address: LUCAS & MERCANTI, LLP, 475 PARK AVENUE SOUTH, 15TH FLOOR, NEW YORK, NY, 10016, US

(73) Assignee: Konica Minolta Medical & Graphic, Inc.

(21) Appl. No.: 11/402,713

(22) Filed: Apr. 12, 2006

(30) Foreign Application Priority Data
Apr. 21, 2005 (JP) JP2005-124070

(51) Int. Cl. A61K 49/04; A61K 9/127

(52) U.S. Cl. 424/9.45; 424/450

(57) ABSTRACT

A method of manufacturing a liposome-containing preparation is disclosed, comprising (a) mixing one or more constituents of a liposome membrane, an aqueous solution of a water-soluble chemical and supercritical carbon dioxide at a temperature of 32 to 65° C. in a pressure vessel, and (b) evacuate the carbon dioxide to form an aqueous dispersion of liposomes enclosing an aqueous solution of a water-soluble chemical, wherein the constituents include at least one phospholipid exhibiting a transition temperature.

This application claims priority from Japanese Patent Application Nos. JP2005-124070 filed on Apr. 21, 2005 which is incorporated hereinto by reference.

### FIELD OF THE INVENTION

The present invention relates to a method of manufacturing liposome-containing pharmaceutical preparations which contain liposomes exhibiting high enclosure ratio of a drug, and liposome-containing pharmaceutical preparations obtained thereby.

### BACKGROUND OF THE INVENTION

Liposomes are closed vesicles having a lipid bilayer (liposomal membrane) formed mainly of phospholipid and having a structure which functions similar to living membrane, which have been noted so far. Liposomes can construct a so-called capsule structure in which water-soluble chemical substances are included in the internal aqueous phase and oil-soluble chemical substances are retained in the interior of the bimolecular layer Active studies have been made studies of application to a drug delivery system (DDS).

Hitherto, there has been employed the Bangham method or a reverse phase vaporization method (also denoted as the REV method) to prepare drug-enclosing liposomes. In these methods, drugs are enclosed within liposomes exhibiting high safety as raw materials and appropriate degradability in vivo, whereas it is necessary to use organic solvents as a solvent for phospholipid forming the liposomal membrane in the process of preparing liposomes. However, the thus obtained liposome-containing pharmaceutical preparation still contains a remained organic solvent and problems arise with characteristics and stability of liposomes, as described in JP-B No. 2619037 (hereinafter, the term, JP-B refers to Japanese Patent Publication). They are not yet in the practical use, based on the reason that remained solvent is toxic, as described in JP-A No. 7-316079 (hereinafter, the term, JP-A refers to Japanese Patent Application Publication).

Further, conventional methods are difficult to allow a sufficient amount of drugs to be enclosed in the liposomes so that problems arise which necessitate dosing of relatively large amounts of the liposome-containing pharmaceutical preparation, imposing an excessive burden on the patient. Taking into account application to contrast medium for diagnosis of which dose becomes large, compared to drugs for medical treatments, there has been desired a liposome-containing pharmaceutical preparation exhibiting high retention efficiency (enclosure ratio) of contrast medium material.

JP-A No. 2003-119120 discloses a method of manufacturing liposomes by using supercritical carbon dioxide instead of organic solvents. This method which is feasible to set various manufacturing conditions can achieve enhanced retention efficiency (enclosure ratio) of material to be included more easily than the conventional method of manufacturing a liposome. However, the use of supercritical carbon dioxide, as disclosed in the foregoing patent document enables preparation of liposomes having enhanced enclosure of water-soluble chemicals, compared to the conventional methods but still further enhancement of the enclosure ratio has been desired. A high enclosure ratio of enclosed material can attain a desired effect even if a small amount of liposomes are brought in vivo, which is expected as a method of manufacturing a preparation for DDS (drug delivery system). However, the use of solubilizing aids such as ethanol is desired in mixing supercritical carbon dioxide with a lipid and a material to be enclosed, resulting in difficulty in manufacture of liposomes of a high enclosure ratio without using any organic solvent, as described in Pharm. Tech. Japan vol. 19, No. 5, page 91-100 (2003). It needs to be taken into account that even if a pharmaceutical chemical is enclosed in liposomes, a remained solubilizing aid lowers membrane strength and the chemicals may leak out with the elapse of time. Accordingly, there are still required not only a method for allowing a pharmaceutical chemical to be efficiently enclosed in the interior of liposomes but also a pharmaceutical form capable of maintain aging stability and enhancing blood retention capability, improvement of preparation composition and enhanced safety of the preparation.

### SUMMARY OF THE INVENTION

In view of the foregoing, it is an object of the present invention to provide a method for manufacturing liposomes having a water-soluble chemical enclosed efficiently and a preparation containing the liposomes. Specifically, a preparation containing a nonionic iodine compound as the water-soluble chemical is a radiographic contrast medium exhibiting superior representation of cancer tissue and enhance safety having easy dischargeability.

Thus, one aspect of the invention is directed to a method of manufacturing a liposome-containing preparation comprising the steps of (a) mixing one or more constituents of a liposome membrane, an aqueous solution of a water-soluble chemical and supercritical carbon dioxide at a temperature of 32 to 65° C. in a pressure vessel, and (b) evacuate the carbon dioxide to form an aqueous dispersion of liposomes enclosing an aqueous solution of a water-soluble chemical, wherein the constituents include at least one phospholipid exhibiting a transition temperature. In one preferred embodiment of the invention, the method comprises (i) mixing one or more liposome membrane constituents and supercritical carbon dioxide in a pressure vessel at a temperature of 32 to 65° C. to form a suspension, (ii) adding an aqueous solution of a chemical to obtain a mixture, and (iii) evacuating the vessel to discharge the carbon dioxide from the mixture to form an aqueous dispersion of liposomes enclosing the water-soluble chemical, wherein the liposome membrane constituents include a phospholipid exhibiting at least a transition temperature.

In another preferred embodiment of the invention, the method comprises (i) supplying liquefied carbon dioxide into a pressure vessel containing a suspension obtained by mixing at least one at least one liposome membrane constituent and an aqueous solution of a chemical, (ii) applying pressure to the vessel at a temperature of 32 to 55° C. to bring the liquefied carbon dioxide to a supercritical carbon dioxide, and (iii) evacuating the vessel to discharge the carbon dioxide to form an aqueous dispersion of liposomes enclosing the water-soluble chemical, wherein the liposome membrane constituents include a phospholipid exhibiting at least a transition temperature. The suspension may be one which is manufactured by mixing the liposome membrane constituents and then supplied into the pressure vessel.

The transition temperature is preferably from 22 to 60° C. After the third step (iii), the obtained aqueous dispersion may be incubated for 0.1 to 3.0 hr. at a temperature within the range of the transition temperature of the phospholipid of the liposome membrane constituents and a temperature of the transition temperature plus 10° C.

The third step (iii) may be followed by the fourth step of filtering the aqueous dispersion of liposomes enclosing the water-soluble chemical under a pressure of 0.01 to 0.8 Mpa at 50 to 90° C., using a hydrostatic extrusion apparatus provided with a membrane filter having a pore size of 0.1 to 1 μm to obtain liposomes having average vesicular size of 0.05 to 0.8 μm.

After the foregoing third step or fourth step, the aqueous dispersion of liposomes enclosing the water-soluble chemical is preferably subjected to ultrafiltration to perform concentration. After the ultrafiltration, the aqueous dispersion may further be subjected to vapor sterilization at 115 to 140° C.

The ultrafiltration can enhance the enclosure ratio of a water-soluble chemical in the liposomes to 25% to 35%. Preferably, the concentration (mol/l) of a water-soluble chemical contained in a water phase preferably is substantially equal between the interior and exterior of the liposome membrane, that is, the concentration (1) of a water-soluble chemical contained in a water phase outside of liposome vesicles is substantially equal to the concentration (2) of a water-soluble chemical contained in a water phase inside the liposome vesicles. Specifically, the ratio of concentration (1) to concentration (2) is preferably within the range of 0.95 to 1.05.

At least 70% of the liposomes manufactured as above is preferably accounted for by vesicles having a membrane formed of two to ten lamellas, that is, 2- to 10-lamella vesicles. The liposome membrane constituents include at least a phospholipid, a cationic lipid, a polyethylene glycol (PEG) group containing lipid (also denoted as PEG-lipid) and sterols, and the molar ratio of phospholipid (excluding PEG-phospholipid)/sterols is preferably from 100/60 to 100/90 and the molar ratio of phospholipid (excluding PEG-phospholipid)/polyethylene glycol group-containing lipid is preferably from 100/5 to 100/25.

The preferred embodiments of invention include a preparation containing liposomes manufactured by the foregoing method, and in preferred embodiments of the invention the water-soluble chemicals are a nonionic iodine compound and a physiologically acceptable auxiliary additive and a liposome-containing preparation used as a radiographic contrast medium is also included.

### DETAILED DESCRIPTION OF THE INVENTION

In the invention, the supercritical state includes a subcritical state. The liposome membrane is also called a lipid membrane. The expression, “being enclosed within liposomes or liposome membrane” means being included in the liposome membrane or liposome and associated with the lipid membrane, or existing in a water phase (internal water phase) enclosed in the interior of the lipid membrane. In the specification, “cancer” refers to a malignant tumor and it is also simply referred to as “tumor”.

In the manufacturing method of liposome-containing preparations according to the invention, liposomes can be manufactured by using supercritical carbon dioxide as a mixing medium (hereinafter, also denoted as a supercritical carbon dioxide method), substantially without using any solubilizing aid. In that case, water-soluble chemicals and/or preparation aids are enclosed within a liposome membrane. In one embodiment of the invention, constituents of the liposome membrane include a phospholipid exhibiting a transition temperature. In the following, there will be described enclosed water-soluble chemicals, liposome membrane constituents, a manufacturing method of liposomes, a manufacturing method of a liposome-containing preparation and a radiographic contrast medium containing liposomes.

A liposome-containing preparation, in which water-soluble chemicals are enclosed within lipid membrane, can be made in the manufacturing method of liposomes of the invention. The water-soluble chemicals are not specifically limited and include materials used as a medicine. Examples of water-soluble chemicals include contrast medium compounds, anticancer materials, an antifungal material, an antioxidant material, antibacterial materials, anti-inflammatory materials, circulation-promoting materials, skin-whitening materials, rough skin-preventing materials, aging prevention materials, new hair growth promotion materials, moisturizing materials, hormone drugs, vitamins, dyes and proteins.

Liposome-containing preparations of the invention are preferably employed as a contrast medium or an anticancer material, and more preferably as a contrast medium. Specifically preferred contrast medium material is a water-soluble nonionic iodine compounds. Preferred examples of such an iodine compound include iomeprol, iopamidol, iohexol, iopromide, ioxilane, iotasul, and iodixanol. These compounds may be used singly or in combination thereof.

In a drug delivery system (DDS) of pharmaceutical material, inclusion in liposomes reduces adverse effects and benefits resulting from enclosure are further increased so that preparation is often made by removing pharmaceutical material which has not been enclosed. In fact, it was reported that even if liposomes achieving approximately 100% enclosure were separated, enclosed components of a liposome suspension preparation leaked out of the liposomes with the elapse of time (Batageri, G. W., Drug Devel. Ind. Pharm. 19, 531-539 (1993)). Such phenomenon is based on instability of the liposome structure, caused by an osmotic pressure effect. When a radiographic contrast medium having a contrast medium material enclosed only in the interior of liposomes, such as a liposome preparation disclosed in WO88/09165, was subjected to an autoclave treatment, the contrast medium material leaked out, as described in JP-A No. 7-316079. There was also discussed a diagnostic significance of a preparation containing unenclosed, free contrast medium material (published Japanese translation of PCT International Publication for Patent Application No. 9-505821). This arises from an usage embodiment inherent to the contrast medium and distinguished from the position that a pharmaceutical compound which is not enclosed in the liposome is of no use from expected results.

A radiographic contrast medium, which is one embodiment of liposome-containing preparations of the invention, usually contains an iodine compound enclosed in liposomes. In such a contrast medium, the proportion of a contrast medium material enclosed in the liposomes, that is, enclosure ratio must be taken into account. The radiographic contrast medium of the invention includes a feature that 65% to 80% by weight of the foregoing water-soluble iodine compound is not enclosed in liposomes and exists in an aqueous medium suspending the liposomes. A preparation in which nearly all or almost of the iodine compound is enclosed in liposomes may be feasible. However, such a preparation is not superior in practice in view of osmotic pressure difference, the form and stability of liposomes, the enclosure ratio and capability as a contrast medium.

To achieve efficient enclosure of an iodine compound and to prevent aging instability of liposomes having the iodine compound, the amount of the iodine compound to be enclosed in the liposomes is limitative in the liposome-containing contrast medium of the invention. Thus, the radiographic contrast medium preferably encloses 10% to 35% by weight of the total amount of an iodine compound, more preferably 10% to 30% and still more preferably 15% to 25% by weight. If an iodine compound enclosed in liposomes accounts for 5% to 30% by weight (preferably 5% to 25%) of the whole, the remaining amount of 70% to 95% (preferably 75% to 95%) by weight which leaked into the aqueous dispersion outside the liposomes, may be substantially disregarded. Microencapsulation of the iodine compound or an osmotic pressure effect of liposomes can prevent instability, leading to enhanced aging stability of a contrast medium material in liposomes. This means that even in a liposome-containing contrast medium, the enclosure ratio of an iodine compound at the time of manufacture and that at the time of using are maintained at the same level, which is preferable in terms of quality control. Lowered enclosure ratio in liposomes during storage results in different preparations depending on the storage term, affecting contrast medium performance.

A liposome-containing preparation manufactured by the method according to the invention is composed of liposomes in which water-soluble chemicals are included in an aqueous phase outside and inside the lipid membrane and substantially containing no organic solvent. Thus, water-soluble chemicals can be efficiently delivered to the targeted organ or focus of tissue in an capsulated form of being enclosed within liposomes serving as a micro-carrier. Specifically, the liposomes used in the invention preferably has a form or structure suitable as suitable liposomes used for a contrast medium. Such liposomes, of which a lipid membrane is composed of several lamellas, exhibit improved aging stability and blood stability, and further raise the enclosure ratio of contrast medium material to enhance contrast medium performance.

Appropriate design of the particle size and lipid membrane of liposomes enclosing water-soluble chemicals can provide targeting function to the liposome-containing preparation. Specifically, in the case of general dosage, it is desirable to take into account both passive targeting and active targeting. The former can control biological behavior through adjustment of vesicle size, lipid composition or charge of the liposomes. Adjustment of the liposome vesicle size within a narrow range can be easily accomplished by the method to be described later. Design of the liposome membrane surface can be achieved by varying the kind and composition of phospholipids and included materials to provide desired characteristics. There should be also examined the introduction of active targeting which enables higher integration and delivery selectivity of the liposomes. For example, introduction of a polymer chain of polyalkylene oxide or polyethylene glycol (PEG), which can control the guidance process to the targeted region, is extremely beneficial.

Any liposomes which do not reach the targeted cancerous tissue or the diseased region is excreted without being accumulated in the normal region, before causing adverse effects by the degradation of the liposome. This can be attained by optimal control of stability of liposomes in relation to the external discharge time in the design of the liposome. Such control of clearance can expect another beneficial effect of the DDS (or Drug Delivery System) chemical form by allowing water-soluble chemicals exhibiting more or less adverse effects in a free form to be enclosed in liposomes. For instance, a water-soluble nonionic iodine compound enclosed in liposomes is deposited non-specifically into a liver, spleen or kidney, preventing difficulty of falling into the situation requiring a lot of time for decomposition and elimination. Retention in vivo can prevent harmful effects or undesirable side effects.

Constituents forming the liposome lipid membrane include a phospholipid, a glycolipid, sterols, glycols, a cationic lipid and a polyethylene glycol group-containing lipid (e.g., PEG-phospholipid). In general, phospholipid and/or glycolipid are preferably used as a lipid membrane constituent of liposomes included in the liposome-containing preparation of the invention. Preferred examples of a neutral phospholipid include lecithin and lysolecithin obtained from soybean or egg yolk, and their hydrogenation products or hydroxide derivatives.

Further, examples of phospholipids include phosphatidylcholine, phosphatidylserine, phosphatidylinositol, phosphatidylglycerol, phosphatidylethanolamine, and sphingomyelin, which are derived from egg yolk, soybeans or other plants and animals, or are semi-synthetically obtainable; phosphatidic acid, dipalmitoylphosphatidylcholine (DPPC), distearoylphosphatidylcholine (DSPC), dimyristoylphosphatidylcholine (DMPC), dioleylphosphatidylcholine (DOPC), dipalmitoylphosphatidylglycerol (DPPG), distearoylphosphatidylserine (DSPS), distearoylphosphatidylglycerol (DSPG), dipalmotoylphosphatidylinositol (DPPI), distearoylphosphatidylinositol (DSPI), dipalmitoylphosphatidic acid (DPPA) and distearoylphosphatidic acid (DSPA), which are synthetically obtainable.

Phospholipids constituting a lipid membrane forming a liposome desirably include a phospholipid exhibiting a transition temperature. The (phase) transition temperature of a phospholipid refers to the temperature at which the phospholipid causes a phase transition between gel and liquid crystal states. The measurement thereof is performed through differential thermal analysis using a differential scanning calorimeter (DSC). Examples of a phospholipid exhibiting a phase transition temperature within the range of 22 to 60° C. include dimyristoylphosphatidylcholine (phase transition temperature: 23-24° C.), dipalmitoylphosphatidylcholine (41.0-41.5° C.), hydrogenated soybean lecithin (53° C.), hydrogenated phosphatidylcholine (54° C.), and distearoylphosphatidylcholine (54.1-58.0° C.).

Examples of cationic lipids usable in the invention include 1,2-dioleoyloxy-3-(trimethylammonium)propane (DOTAP), N,N-dioctadecylamidoglycylspermine (DOGS), dimethyloctadecylammonium bromide (DDAB), N-[1-(2,3-dioleyloxy)propyl]-N,N,N-trimethylammonium chloride (DOTMA), 2,3-dioleyloxy-N-[2(spermine-carboxamido)ethyl]-N,N-dimethyl-1-propaneaminiumtrifluoroacetate (DOSPA) and N-[1-(2,3-dimyristyloxy)propyl]-N,N-dimethyl-N-(2-hydroxyethyl)ammonium bromide (DMRIE).

Examples of a cationic phospholipid include an ester of phosphatidic acid and an aminoalcohol, for example, ester of dipalmitoylphosphatidic acid (DPPA) or distearoylphosphatidic acid (DSPA) and hydroxyethylenediamine. These cationic lipids are preferably contained in an amount of 0.1% to 5% by weight, more preferably 0.3% to 3% by weight, and still more preferably 0.5% to 2% by weight of the total lipid amount.

These phospholipids may be used alone or in combination of two or more. Two or more charged phospholipids are preferably used in combination of negative-charged phospholipids or in combination of positive-charged phospholipids in term of aggregate prevention of the liposomes. The combined use of a neutral phospholipid and charged phospholipid is preferably in a weight ratio of 200:1 to 3:1, more preferably 100:1 to 4:1, and still more preferably 40:1 to 5:1.

Examples of glyceroglycolipids include glycerolipids such as digalactosyldiglyceride and digalactosyldiglyceride sulfuric acid ester; sphingoglycolipids such as galactosylceramide, galactosylceramide sulfuric acid ester, lactosylceramide, ganglioside G7, ganglioside G6 and ganglioside G4.

In addition to the foregoing lipids, other substances may optionally incorporated as a constituent of the liposome membrane. For example, cholesterol, dihydrocholesterol, cholesterol ester, phytosterol, sitosterol, stigmasterol, campesterol, cholestanol, lanosterol and 2,4-dihydroxylanosterol are cited as a layer stabilizer. Further, sterol derivatives such as 1-O-sterolglucoside, 1-O-sterolmaltoside and 1-O-sterolgalactoside have been shown to be effective in stabilization of liposome (as described in JP-A No. 5-245357) and of the foregoing sterols, cholesterol is specifically preferred.

A cholesterol enclosed in the liposome membrane is capable of functioning as an anchor to introduce a polyalkylene oxide. JP-A No. 9-3093 discloses novel cholesterol derivatives, in which various functional substances can be efficiently fixed at the top of a polyoxyalkylene chain, which can be employed as a liposome constituent.

Sterols are used preferably in a molar ratio of phospholipid (not including PEG-phospholipid)/sterols of 100/60 to 100/90 and more preferably 100/70 to 100/85. The molar ratio is based on the amount of phospholipid which does not include PEG-phospholipid. The PEG-phospholipid refers to a polyethylene glycol-modified phospholipid, which is also denoted as a PEGylated phospholipid. A molar ratio of less than 100/60 cannot achieve stabilized dispersion of the lipid mixture.

In addition to the foregoing sterols, glycols may be added as a constituent of a liposome vesicular membrane. In the preparation of liposome, addition of a phospholipid together with glycols enhances the holding efficiency of a water-soluble iodine compound included within liposome vesicles. Examples of glycols include ethylene glycol, diethylene glycol, triethylene glycol, propylene glycol, dipropylene glycol, trimethylene glycol, and 1,4-butanediol. Glycols are used preferably in an amount of 0.01% to 20% by weight of the total weight of lipids, and more preferably 0.5% to 10% by weight.

In response to the intended objective of the liposome-containing preparation of the invention, there may be used a phospholipid or a compound containing a polymer chain of polyalkylene oxide (polyoxyalkylene chain) group (or PAO group) or the like, as a constituent of the liposome membrane. Attachment of a polyalkylene oxide group or polyethylene glycol (PEG) group onto the liposome membrane surface can overcome instability of the liposome itself, such as destruction or aggregation, and aging stability can also be improved. There can also be provided a new function to the liposome. For example, such a PEG-modified liposome can be expected to have the effect of becoming less recognizable from an immune system. It was proved that a liposome having a hydrophilic tendency increased blood stability and thereby the concentration in blood can be more stably maintained over a long period of time, as described in Biochim. Biophys. Acta., 1066, 29-36 (1991). Accordingly, adjustment of the length of an oxyethylene unit of a PEG group, represented by —(CH₂CH₂O)ₙ—H and the proportion to be introduced can control its function. Polyethylene glycol having 10-3500 oxyethylene units (preferably 100-2000 oxyethylene units), as a PEG group, is suitable. Polyethylene glycol is preferably contained in an amount of 1% to 40% by weight, and more preferably 5% to 25% by weight, based on the lipid constituting the liposome. PEG-modification of a liposome can be accomplished using commonly known techniques. Preferably, PEG-phospholipid may be used as a lipid having a polyethylene glycol group. As described later, it exhibits an action such as a solubilizing aid when mixing a phospholipid with supercritical carbon dioxide.

In place of polyethylene glycol (PEG), commonly known polyalkylene oxide groups may be introduced, which is represented by general formula: -(AO)ₙ—Y, wherein AO is an oxyalkylene group of 2 to 4 carbon atoms, n represents a mean addition molar number and is a positive number of 1 to 2000 (preferably, 10 to 500, and more preferably 20 to 200); and Y is a hydrogen atom, an alkyl group or a functional group. Examples of an oxyalkylene group having 2 to 4 carbon atoms include an oxyethylene group, oxypropylene group, oxytrimethylene group, oxytetramethylene group, oxy-1-ethylethylene group and oxy-1,2-dimethyletthylene group.

When n is 2 or more, plural oxyalkylene groups may be the same or differ. In the latter case, differing oxyalkylene groups may be in a random form or in a block form. To provide hydrophilicity to an oxyalkylene group, ethylene oxide alone is preferably addition-polymerized, in which n is preferably 10 or more. In cases when different alkylene oxides are addition-polymerized, it is desirable that at least 20 mol % (preferably at least 50 mol %) of ethylene oxide is addition-polymerized. To provide lipophilicity to an oxyalkylene group, it is preferred to increase the molarity of alkylene oxide(s) other than ethylene oxide. For example, a liposome containing a block copolymer of polyethylene oxide and polypropylene oxide (or polyethylene oxide-block-polypropylene oxide) is one preferred embodiment of this invention.

The functional group of the foregoing Y is to attach functional material such as sugar, glycoprotein, antibody, lectin and a cell adhesion factor to the top of a polyalkylene oxide group and examples thereof include an amino group, oxycarbonylimidazole group and N-hydroxysuccinimide. The liposome anchoring a polyalkylene oxide chain, to the top of which the foregoing functional material is bonded, not only exhibits desired effects due to introduction of a polyalkylene oxide group but also gives full play of functions of the functional material, for example, a function as a recognition element, such as directivity to a specific organ (namely, organotropism) and cancer tissue directivity. To provide cancer tissue directivity, an antitumor monoclonal antibody corresponding to tumor-specific antigen existing in oncocyte, as a functional material, is attached to liposome membrane to form a liposome exhibiting enhanced target selectivity (as described, for example, in JP-A No. 11-28087).

Introduction of a polyalkylene oxide chain to the liposome membrane can be achieved by employing commonly known techniques. Phospholipids or compounds containing a polyalkylene oxide chain may be used singly or in combination of at least two kinds thereof. The content is usually 0.001 to 50 mol %, preferably 0.01 to 25 mol %, and more preferably −0.1 to 10 mol %, based on the total amount of liposome membrane constituents.

Other compounds which may be added include a phosphoric acid dialkyl ester such as dicetyl phosphate, as a negative-charged material and an aliphatic amine such as stearylamine as a positive-charged material.

The vesicular particle size (or liposome vesicle size, hereinafter, also denoted simply as vesicle size) of the liposomes, as microparticles and its distribution are closely correlated with enhanced blood retention property, targeting ability and delivery efficiency which are aimed in the invention. The vesicle size can be determined in the manner that a dispersion containing liposome vesicles enclosing an iodine compound is frozen and fractured, following which carbon is vapor-deposited onto the fractured interfaces and the deposited carbon is observed with an electron microscope (freeze fracture TEM method). In the invention, the average particle size refers to a simple (or arithmetic) average of the given number of observed vesicular particles of the contrast medium, for example, 20 vesicles. This value usually agrees with or is close to the central vesicle size which refers to a vesicle size having the highest frequencies in the vesicle size distribution.

Sizing of the liposome particles is important to enhance active targeting capability of the liposome. For example, Japanese Patent No. 2619037 discloses that unfavorable retention in lung capillaries can be avoided by removal of liposome of 3 μm or more. However, liposome of 0.5 to 3 μm does not necessarily exhibit tumor directivity. In the liposome-containing preparation of the invention, liposomes are manufactured by adjusting the average particle size to be within the range of 0.05 to 0.8 μm in response to the use thereof. In the case of a radiographic contrast medium for photographing liver, for example, the average liposome particle size is preferably 0.2 to 0.8 μm. Liposomes enclosing a water-soluble iodine compound and falling within the foregoing particle size range become a target for capture phagocytosis by reticular cells which exist mostly in non-tumor tissue and clearly contrast with tumor tissue. In liposomes used for a contrast medium for liver, PEG on the liposome surface is preferably as little as possible.

To make liposomes tumor directive, based on EPR effect (enhanced permeability and retention) employing the bloodstream, the average particle size of liposomes is 0.1 to 0.2 μm, and preferably 0.11 to 0.13 μm. An average particle size of 0.11 to 0.13 μm can render it possible to concentrate the liposome-containing preparation selectively onto cancer tissue.

There have been proposed various methods for preparing liposomes. Different methods often finally lead to liposomes extremely differing in form and characteristics, as described in JP-A No. 6-80560. Therefore, a method is optimally chosen in accordance with the form or characteristics of a desired liposome. In general, a liposome is prepared by dissolving lipid components such as phospholipid, sterol and lecithin, almost without exception, in organic solvents such as chloroform, dichloromethane, ethyl ether, carbon tetrachloride, ethyl acetate, dioxane or tetrahydrofuran (THF). Specifically, chlorinated solvents are often employed. The thus prepared liposome necessarily contains an organic solvent.

To manufacture liposomes used in the invention, a preparation method using supercritical or subcritical carbon dioxide is employed based on the reasoning that carbon dioxide is suitable because it exhibits a critical temperature of 31.1° C. and a critical pressure of 75.3 kg/cm² and can be relatively easily handled, is an inert gas and non-toxic in the human body even when retained, and a high-purity liquid is inexpensively and easily available. However, even if no organic solvent was used in the conventional supercritical carbon dioxide method, it was recommended to use ethanol or the like to perform efficient dispersion of lipids in supercritical carbon dioxide, for example, as described in JP-B No. 2619037. Accordingly, a treatment of a multi-step process over a long time is required to remove residual organic solvents. It is difficult to completely remove residual organic solvents, specifically, chlorinated organic solvents, which is a concern for adverse effects on organisms.

The manufacturing method of the invention was developed so as to manufacture liposomes having a form or structure suitable for a radiographic contrast medium which is dosed at a relatively high dosage, compared to conventional drugs. Liposomes manufactured by a method using supercritical carbon dioxide contains substantially no organic solvent such as chlorinated organic solvents, ethanol or other organic solvents, and exhibiting preferable characteristics for enclosure of water-soluble chemicals. Thus, there was shown enhancement of liposome formation rate, enclosure ratio of chemicals and retention rate of enclosed chemicals, as compared to the conventional method. Further, the method of this invention is applicable even on an industrial scale. The foregoing expression, substantially no organic solvent means that the residual organic solvent content in the liposome-containing preparation is not more than 10 μg/l.

The first and second steps of the manufacturing method of liposomes of the invention include two ways based on differences in the order of mixing. Thus, one way comprises: (i) the first step of mixing liposome membrane constituents and supercritical carbon dioxide to obtain a suspension and (ii) the second step of adding an aqueous solution of chemicals to the suspension. Another way comprises: (i) the first step of supplying liquefied carbon dioxide to a pressure vessel containing a suspension obtained by mixing liposome membrane constituents and supercritical carbon dioxide and (ii) the second step of applying pressure to the interior of the pressure vessel with heating at a temperature from 32 to 55° C. with mixing the suspension and the liquefied carbon dioxide to convert the liquefied carbon dioxide to supercritical carbon dioxide.

The manufacturing method further comprises the following third and fourth steps. Thus, the method comprises: (iii) the third step of reducing the pressure within the vessel (or evacuating the vessel) to discharge carbon dioxide to form an aqueous dispersion of liposomes enclosing the chemicals (preferably together with a pharmaceutical additive) inside liposomes, followed by (iv) the fourth step of filtering the aqueous dispersion of liposomes enclosing the chemicals by a hydrostatic extrusion apparatus provided with a filter membrane with pores having a size of 0.1 to 1 μm at a temperature of 50 to 90° C. under a pressure of 0.01 to 0.8 Mpa to size the liposomes to obtain liposomes having an average vesicle size of 0.05 to 0.8 μm.

After the third and fourth steps, the aqueous dispersion of liposomes enclosing the chemicals may be subjected to ultrafiltration to perform concentration. Further, after the ultrafiltration, steam sterilization may be conducted at 115 to 140° C.

After completion of the foregoing steps, there is obtained a liposome-containing preparation in which the molar ratio of phospholipid (not including PEG-phospholipid)/sterols is 100/60 to 100/90, the molar ratio of phospholipid (not including PEG-phospholipid)/polyethylene glycol group-containing lipid is 100/5 to 100/25, and liposomes having lipid membrane formed of two- to ten-lamella account for at least 70%.

The foregoing respective steps will be detailed as follows.

First and Second Steps

In the first and second steps, liposome membrane constituents, liquefied carbon dioxide and an aqueous chemical solution (containing water-soluble chemicals and an additive such as a preparation auxiliary) are mixed within a pressure vessel. The first and second steps include two ways based on the mixing order.

To a pressure vessel, phospholipids as a lipid constituent forming a liposome membrane and a material exhibiting stabilization action onto lipid membrane (hereinafter, also denoted as lipid membrane-stabilizing material) are added, and liquefied carbon dioxide is further added thereto and mixed preferably under strong agitation. Examples of a material exhibiting stabilization action include cationic lipid, lipid containing polyethylene glycol group and sterols. Phospholipids are preferably those including at least one phospholipid exhibiting a transition temperature of 22 to 60° C. Liquefied carbon dioxide is rendered to a supercritical or subcritical state under the pressure and temperature falling within the range to be described later. The liposome membrane constituents and carbon dioxide in a supercritical (or subcritical) state are sufficiently mixed and dissolved or dispersed. Alternatively, to a pressure vessel containing liquefied carbon dioxide, these compounds may be added and mixed, followed by being rendered to the supercritical state by adjustment of temperature and pressure. Subsequently, to the thus formed supercritical carbon dioxide containing the phospholipids and the lipid membrane-stabilizing material, an aqueous solution containing water-soluble chemicals to be enclosed, such as a nonionic iodine compound and pharmaceutical additives, is introduced thereto to form micelles.

Alternatively, to a pressure vessel containing a suspension obtained by mixing phospholipid as a lipid constituent forming a liposome membrane and at least one of cationic lipid, lipid containing polyethylene glycol group and sterols and an aqueous solution containing water-soluble chemicals and a pharmaceutical additive, liquefied carbon dioxide is supplied preferably under strong agitation. Subsequently, the mixture is heated with applying pressure to render liquefied carbon dioxide to a supercritical state and is further mixed under strong agitation to form micelles. The suspension is obtained by mixing liposome membrane-forming constituents and an aqueous solution containing water-soluble chemicals in the pressure vessel. Alternatively, such a suspension is prepared separately and then supplied into the pressure vessel.

Efficiency of enclosing material to be enclosed within liposomes depends on the total amount of lipid forming liposome membrane, the ratio of lipids to supercritical carbon dioxide and the ratio of lipids to an aqueous solution containing material to be enclosed. The total amount of lipids refers to the total weight of liposome membrane constituting materials such as phospholipids, sterols and other lipids. To allow almost all liposomes to be formed as several-lamellar liposomes (or vesicles) rather than a unilamellar liposomes (or esicles), a lipid may be added in an amount of 1.5 to 2.5 times the conventional amount. More specifically, 0.075 to 0.125 (preferably 0.08 to 0.1) part by weight of lipid, based on 1 part by weight of carbon dioxide is mixed under strong agitation. An excessive amount of lipids causes may remain lipid undissolved. However, a lipid phase containing the foregoing lipid may exist at the initial stage of stirring. Any following strong agitation causes a large number of CO₂/water interfaces, whereby a number of small micelles are formed, leading to an increase of the enclosure ratio. Emulsification of lipids in supercritical carbon dioxide can be promoted in the presence of a PEG group-containing lipid, as described below, even without addition of ethanol or the like.

Strong agitation, the preferred range of which differs depending on the volume of the mixed solution and stirring means, means that, for example, a 10 to 100 ml solution is stirred using a nearly cylindrical stirring rod of 15 mm length and 5 mm diameter and a magnetic stirrer at a rate of 400 to 4,000 rpm (preferably 1,000 to 1,500 rpm). Even if the volume of the mixed solution or stirring means differ from the foregoing, it is preferred to set appropriate stirring conditions taking into account the shearing force which is applied to the solution under the foregoing stirring conditions. Specifically, stirring conditions preferably satisfy the following requirement:
C=N×V^{-0.15}
where C is the number of revolutions (rpm), V is the volume of the mixed solution and N is 300 to 3,000. The strong agitation duration, which is optionally set according to the amount of liquid to be mixed and the amount of lipid, is usually from 1 to 120 min., and preferably from 5 to 60 min. The foregoing strong agitation duration includes the time for supplying the aqueous solution of chemicals.

Mixing liposome membrane constituents and supercritical carbon dioxide over a prescribed period of time under strong agitation under the foregoing conditions enhances the liposome formation rate and obtain preparations containing liposomes exhibiting enhanced enclosure ratio of chemicals. Thus, strong agitation under such conditions promotes formation of liposomes and can obtain an aqueous dispersion of liposomes exhibiting an enhanced enclosure ratio of chemicals. Stirring means are not specifically limited but conventional stirring machines are usable, such as a magnetic stirrer, a homogenizer, a homomixer, and an ultramixer.

Lipids including phospholipid, cationic lipid and sterol are inherently difficult to dissolve or disperse in supercritical carbon dioxide or water. To achieve efficient formation of liposomes in the desired form, it is an important condition that lipid is well dispersed in supercritical carbon dioxide and that emulsification proceeds to form a homogeneous state. Accordingly, it is preferred to perform solution, dispersion or mixing by use of a compound containing a hydroxyl group.

The compounds containing a hydroxyl group (or hydroxyl group-containing compounds) described above include compounds which contain, as a hydrophilic group, a hydroxy group, a polyol group, a polyalkyleneglycol ether group, or a combination of polyol/polyglycol ether group. The hydroxyl group containing compound usable as a solubilizing agent is preferably one which exhibits affinity with lipid membrane constituents such as a phospholipid or cholesterol and are easily miscible with it. Amphiphilic ones which exhibit optimal hydrophilicity and lipophilicity are suitable to disperse lipid membrane constituents in polar carbon dioxide fluid (or liquid).

When there is concern of safety regarding toxicity of a residual solubilizing agent, it is desirable not to use lower alcohols as the hydroxyl group-containing compound. Taking into account effectiveness and safety, the solubilizing agent is preferably a compound containing a polyethylene glycol group, and more preferably a lipid containing a polyethylene glycol group, in which is preferred a polyethylene glycol group having an oxyethylene unit of 10 to 3500 (preferably 100 to 2000).

The use of one or more hydroxyl group-containing compounds enhances the enclosure ratio. Hydroxyl group-containing compounds are used as a solubilizing agent, preferably in an amount of 0.01% to 1% by weight of supercritical or subcritical carbon dioxide and more preferably 0.1% to 0.8% by weight. In the case of the hydroxy group-containing compound being a polyethylene glycol group-containing lipid, the molar ratio of phospholipid (not including PEG-phospholipid)/polyethylene glycol group-containing lipid is usually 100/5 to 100/25, and preferably 100/5 to 100/10.

In the conventional supercritical carbon dioxide method using ethanol or the like as a solubilizing aid, ethanol promotes dissolution or dispersion of lipids in supercritical carbon dioxide. As a result, a two-phase system comprised of a carbon dioxide region containing cholesterol, phospholipid and the like and a water region mainly containing PEG-modified phospholipid and water-soluble chemicals is formed, which is stirred to promote emulsification of supercritical carbon dioxide to form lipid micelles. Finally, there are predominantly formed liposomes of a uni-lamellar lipid membrane.

Contrarily, in the invention, a polyethylene glycol group-containing lipid (for example, polyethylene glycol-modified phospholipid, which is also denoted as PEG-phospholipid) promotes emulsification insteade of a solubilizing aid such as ethanol. Water-soluble chemicals and a PEG-phospholipid are dissolved or dispersed in an aqueous phase, not only lowering the surface tension of water but also forming a phase of accumulated phospholipid and cholesterol, and forming the state of a three-phase system with supercritical carbon dioxide containing nearly no lipid. Stirring promotes emulsification of supercritical carbon dioxide and forms far more interfaces than conventional methods. Lipids are disposed onto such a large number of interfaces to form a lipid monolayer. Specifically when strong agitation, as described above, is performed in an increased amount of lipid, a number of small homogeneous micelles are formed.

Even if ethanol or the like is not present, emulsification of supercritical carbon dioxide is caused, forming lipid micelles. Further in the method of the invention, the strong agitation efficiently forms minute homogeneous micelles. In the subsequent step of forming a lipid bilayer from a lipid monolayer, induced by discharge of carbon dioxide, the enclosure ratio of water-soluble chemicals is enhanced, for example, from 17% to 21%. The conventional supercritical carbon dioxide method using ethanol or the like forms mainly unilamellar liposomes. On the contrary, the amount of lipid is increased in the method of the invention, resulting in formation of multilamellar liposomes of two-lamellar to ten-lamellar membrane. It is to be noted that change in lipid amount, stirring conditions and solubilizing aids results in an enhanced enclosure ratio and formation of liposomes of a different form. Further in such multilamellar liposomes, a surprising increase in enclosure ratio is observed in the sizing step, suggesting occurrence of rearrangement of the membrane.

In the manufacturing method of the invention, the temperature of supercritical carbon dioxide (subcritical carbon dioxide inclusive) is set usually at 32 to 70° C., preferably at 32 to 65° C., and more preferably at 45 to 65° C. In cases when liposome membrane constituents include a phospholipid exhibiting a transition temperature, the temperature is set preferably at a temperature of not more than a temperature of the phase transition temperature plus 10° C., more preferably not more than a temperature of the phase transition temperature plus 5° C., and still more preferably a temperature nearly equivalent to the phase temperature.

Hitherto, heating to a temperature higher than the phase transition temperature of a phospholipid brings a phospholipid having a phase transition temperature to the liquid crystal state, whereby fluidity is enhanced and the phospholipid is efficiently mixed with the supercritical carbon dioxide, facilitating liposome preparation, therefore, manufacturing of liposomes was conducted at 50 to 80° C. However, it was discovered by the inventors that when the temperature of supercritical carbon dioxide is brought to a temperature near the phase transition temperature of the phospholipid, the phospholipid is not excessively heated and causing no denaturation and resulting in a regular arrangement of phospholipids to form a liposome membrane. Under the condition of performing string agitation to promote emulsification, it is desirable to accept the foregoing temperature range even in terms of reducing local overheating. The suitable pressure applied to supercritical carbon dioxide is optionally chosen from the foregoing temperature range but preferably is 5 to 50 MPa, and more preferably 10 to 30 MPa.

Third Step

In the third step, after sufficiently mixing liposome membrane constituents, supercritical carbon dioxide and an aqueous solution of water-soluble chemicals, water may optionally be added thereto and the interior of the pressure vessel is evacuated. An aqueous dispersion of liposomes enclosing water-soluble chemicals and the like is formed by discharging carbon dioxide. In this stage, the liposomes are assumed to be phase-inversed to the water-phase so that only discharging carbon dioxide results in formation of an aqueous dispersion of liposomes enclosing water-soluble chemicals. Since the aqueous solution is enclosed in the interior of the liposomes, a contrast medium material exists not only in the water phase outside the liposomes but also mainly in the water phase within the liposomes, which is in the state of so-called enclosure.

The aqueous liposome dispersion obtained in the third step may be subjected to incubation at a temperature within the range from the transition temperature of a phospholipid of liposome membrane constituents to a temperature of the transition temperature plus 10. C over a period of 0.1 to 3′ hr. and preferably 10 to 60 min. Performing incubation under the foregoing conditions, for instance, promotes separation or dispersion of liposomes in an aggregate form. Performing such an additional operation causes rearrangement of lipid molecules with increased fluidity within the lipid membrane during the subsequent fourth step of the pressurized filtration, leading to enhanced enclosure of water-soluble chemicals as well as formation of a stable membrane structure.

Fourth Step

The particle size of liposomes (or liposome vesicle size) can be controlled by variation of formula or process conditions. An increase of the pressure applied in the supercritical state reduces the particle size of the formed liposomes. To allow liposomes to fall within the narrow particle size distribution, the liposomes may be filtered with a polycarbonate membrane or cellulose type membrane. In the fourth step, an aqueous liposome dispersion obtained by adjusting the interior of the pressure vessel to atmospheric pressure by introducing air (third step) is allowed to pass through membrane filter of plural filtering membranes having a pore size of 0.1 to 1.0 μm. The filtering membrane filter usable in the invention may be a polycarbonate type or a cellulose type. Plural filters are used preferably in the order of a greater pore size to a less pore size and the pore size is finally lessened preferably to 0.05 to 0.4 μm, more preferably 0.1 to 0.4 μm, and still more preferably 0.15 to 0.2 μm. Pressurized extrusion filtration is operated at a temperature of 50 to 90° C. (preferably 55 to 85° C.) under the pressure of 0.01 to 1.0 MPa (preferably 0.01 to 0.8 MPa). Heating to a temperature higher than the phase transition temperature of a phospholipid brings a phospholipid having a phase transition temperature to the liquid crystal state, whereby fluidity is enhanced. In the manufacturing method of the invention, since phospholipids forming a lipid membrane of liposomes include at least a phospholipid exhibiting a transition temperature, liposomes with a narrow size distribution can be obtained without causing clogging of filters, even when using a relatively viscous dispersion of liposome enclosing water-soluble chemicals.

Operation for sizing is performed in a hydrostatic extrusion apparatus provided with a membrane filter of 0.1 to 1 μm pores. Thus, liposomes are forced through a filter using various commercially available hydrostatic extrusion apparatuses, such as Extruder (trade name, produced by Nichiyu Liposome Co.) and Liponizer (trade name, produced by Nomura Microscience). Liposomes of a narrow size distribution can be efficiently manufactured by use of such hydrostatic extrusion apparatus. To perform sizing at a less average particle size level, filtration is conducted, for example, using a filter of 0.45 μm or the like. Pressurized filtration may be repeated. Extrusion filtration methods are described in, for example, Biochim. Biophys. Acta 557, 9 (1979). Introduction of the extrusion operating step was found to be advantages, such as enhancement of the enclosure ratio of water-soluble chemicals, in addition to sizing described above. The use of lipids at an amount more than usual one results in formation of more multilamellar liposomes than unilamellar liposomes, as reported in, for example, Pharm. Tech. Japan vol. 19, No. 5, page 91-100 (2003). It is assumed that an outermost layer of multilamellar liposomes easily flakes off in the pressure extrusion operation described above, and reconstruction of the membrane structure and sizing occur concurrently, resulting in formation of liposomes of two- to ten-lamellar liposomes. Subjecting liposomes to the foregoing treatment results in not only enhanced enclosure ratio of material enclosed in liposomes but also formation of liposomes of less particle sizes. There is also no concern of lowering of membrane strength, due to a remained solubilizing aid, leading to superior storage stability of liposomes. In addition to the foregoing enhanced enclosure ratio, advantages such as adjustment of the concentration of a water-soluble chemical existing outside the liposomes, exchange of liposome dispersion and concurrent removal of unwanted materials become feasible. Prior to filtration with a membrane filter having the foregoing pore size, preliminary filtration may be conducted using a membrane filter of approximately 1.0 to 2.0 μm for the purpose of sizing or removal of unwanted material.

Concentration Step

This is a step of concentrating the filtrate containing the thus formed liposomes. An aqueous liposome dispersion is filtered with a membrane filter as above and may optionally be subjected to ultrafiltration, centrifugal separation or gel permeation to remove chemicals not enclosed within the liposome to achieve purification. In the invention, it is preferred to perform concentration by ultrafiltration after completion of the third and fourth steps.

Ultrafiltration is conducted by use of a conventional ultrafiltration membrane and device. An ultrafiltration membrane can employ membranes composed of resin such as acrylonitrile copolymer, aromatic nylon, polysulfon, polyfluorovinylidene, polyethersulfon or polyimide. The cut-off molecular weight of these ultrafiltration membrane is desirably 10,000 to 20,000. Operation of the ultrafiltration is executed in a dead-end filtration system in which recovery is carried out under a temperature of 20 to 30° C. (preferably 25 to 30° C.) and a pressure of 0.01 to 0.5 MPa (preferably 0.1 to 0.45 MPa) or in a cross-flow system. It may also be a centrifugal ultrafiltration which can be rapidly executed and in which filtration is accelerated by centrifugal operation suitable for small volume.

In the manufacturing method of the invention, performing the foregoing filtration can raise an enclosure ratio of water-soluble chemicals into liposomes to the range of 25% to 35%. The enclosure ratio refers to the weight ratio of water-soluble chemicals enclosed in the interior of liposomes to whole water-soluble chemicals. In concentration by ultrafiltration, water-soluble chemicals existing outside liposomes move together with salts and an aqueous medium to the secondary side of the ultrafiltration membrane. In an aqueous medium containing liposomes, having a high molecular weight and incapable of permeating through the filtration membrane, the concentration of water-soluble chemicals decreases and that in the liposomes increases. Ultimately, water-soluble chemicals enclosed in liposomes are maintained inside the liposome, resulting in an increase of the enclosure ratio.

Concentration employing ultrafiltration may be conducted after the third step. This is effective as a concentration treatment for extrusion filtration performed in the subsequent fourth step. Even when such an intermediate concentration treatment is conducted, ultrafiltration is again performed after the fourth step. Alternatively, concentration via ultrafiltration continues until reaching the prescribed liposome concentration and conventional auxiliary additives such as a diluent is optionally added thereto, then, the respective concentrations of liposomes, water-soluble chemicals and the auxiliary additives are finally adjusted. Further, an aqueous liposome dispersion is concentrated, reduced to a lower volume and then subjected to freeze-drying, whereby powdery liposomes can be efficiently obtained. The thus obtained liposomes are suspended in an aqueous medium immediately before the use thereof.

After completion of the foregoing ultrafiltration, liposomes may be subjected to steam sterilization at 115 to 140° C., preferably 118 to 125° C., and more preferably at 121° C. Thereby, an aseptic product is obtained and transferred to the packaging step. Liposomes manufactured by the method of the invention are those which are formed of substantially two- to ten-lamellar membrane, preferably two- to several-lamellar membrane (for example, three-lamellar, four-lamellar, five-lamellar or six-lamellar membrane). Such liposomes are formed as a main component in the manufacturing method comprising the foregoing (i)-(v) steps. The expression “substantially” means that liposomes formed of two-lamellar to ten-lamellar membrane account for at least 70%, preferably at least 80%, and more preferably 80% to 98% of all liposomes contained in the preparation of the invention.

A liposome formed of a one-lamellar membrane is a liposome constituted of a single membrane of a phospholipid bilayer (unilamellar vesicle), the replica of which is recognized nearly as a single layer in transmission electron microscopic (TEM) observation using a freeze-fracture replica technique. Thus, when observing the imprint of particles remaining in the carbon film, one having no difference in level is judged as a unilamellar membrane. On the contrary, one having at least two differences is judged as a multilamellar membrane. Liposomes constituted of a two- or three-lamellar membrane exhibit increased strength, compared to single-lamellar liposomes (or unilamellar vesicles) and are not destroyed even during the sizing treatment conducted in the above-described fourth step.

The single-lamellar liposomes (unilamellar vesicles) can be efficiently prepared using the foregoing supercritical carbon dioxide as a solvent for lipids and by a phase separation via water, specifically using ethanol as a solubilizing aid. Contrary to that, in conventional manufacturing methods of liposomes, for instance, the Bangham method or the reverse-phase evaporation method, liposomes formed of a multi-lamellar membrane and having various sizes and forms (multilamellar vesicles) often account for a large proportion of the whole. Liposomes (or vesicles) of a unilamellar or several-lamellar membrane exhibit the advantage that the dosage of liposomes, that is, the amount of lipid to be dosed is less than multi-lamellar vesicles (MLV).

Of several-lamellar liposomes (several-lamellar vesicles), a unilamellar vesicle as a unilamellar liposome, specifically, a large unilamellar vesicle (LUV) displays the advantage of a larger enclosure capacity than a multilamellar vesicle. However, even uni- or several-lamellar liposomes (vesicles) exhibiting an enhanced enclosure capacity become instable when the weight of an enclosed compound is excessive. Specifically, a tendency of weakness upon rapid change of ionic strength was observed. In manufacturing liposomes used for the preparation of the invention, the supercritical carbon dioxide method and the subsequent sizing process are improved so that liposomes of two- to 10-lamellar membrane structures, and preferably several-lamellar liposomes (vesicles) are efficiently formed. Further, the liposome membrane preferably includes a compound (e.g., phospholipid) containing a polyalkyleneoxide group, sterols, or a glycol to enhance stability of the lipid membrane. The thus manufactured liposomes proved to be stable upon salt shock.

Manufacturing of Liposome-Containing Preparation

The liposome-containing preparation of the invention contains liposomes as described above and can be manufactured using at least one physiologically acceptable pharmaceutical auxiliary additive by techniques known in the art. The preparation contains a pharmaceutical auxiliary additive in a water phase within liposomes and in the aqueous medium suspending the liposomes. The auxiliary additive refers to a compound which is to be added together with the water-soluble pharmaceutical chemicals and further various substances can be employed based on techniques for preparing contrast mediums. Specific examples thereof include physiologically acceptable buffering agents, edetate chelating agents such as EDTA Na₂—Ca (or disodium calcium ethylenediaminetetraacetate) or EDTA Na₂ (or disodium ethylenediaminetetraacetate) and optionally, an osmotic pressure-adjusting agent, a stabilizer, an antioxidant such as α-tocopherol, and a viscosity adjusting agent. Water-soluble amine type buffering agents and chelating agents are preferably included. As a pH buffering agent, amine-type buffering agents and carbonate type buffering agents are preferred, of which amine type buffering agents are more preferred and trometamol (also denoted as tromethamine or 2-amino-2-hydroxymethyl-1,3-propanediol) is specifically desirable. Of chelating agents preferable is EDTA Na₂—Ca (edetate calcium disodium).

The foregoing aqueous medium refers to a solvent which is basically composed of water capable of dissolving nonionic iodine compounds or auxiliary additives. There is usable sterilized water containing no exothermic material. When the water-soluble chemicals or auxiliary additives are contained in an aqueous solution other than the water phase (i.e., an aqueous medium suspending the liposomes), the water-soluble chemicals are contained preferably at the same concentration between the interior and exterior of the lipid membrane. Accordingly, no difference in osmotic pressure is caused between inside and outside of the membrane, whereby structure stability of the liposome is maintained. Thus, instability of aged liposomes, due to the osmotic pressure effect can be prevented, leading to enhanced holding stability of water-soluble chemicals inside liposomes.

The pH of the above-mentioned solution or suspension is preferably 6.5 to 8.5 at room temperature, and more preferably 6.8 to 7.8. When contrast medium material is a water-soluble polyhydric iodine compound, a preferred buffer solution is one exhibiting a negative temperature coefficient, as described in U.S. Pat. No. 4,278,654. Amine buffer solution meets such a requirement and preferably is TRIS. Such a type of a buffer solution exhibits a low pH at autoclave temperatures, thereby, stability of a liposome-containing preparation in the autoclave increases and returns to the physiologically acceptable pH at room temperature. It is therefore extremely convenient that a liposome preparation can be subjected to autoclave sterilization to manufacture sterile preparation for injection and storage stability can be secured. The preparation of the invention is marketed in a sterilized form. In that case, a sterile preparation can be obtained by sterile filtration, autoclave sterilization or heat sterilization.

In the invention, the weight of a liposome membrane lipid must be taken into account, in addition to enclosure efficiency of water-soluble chemicals and stability of enclosure. An increase in weight of liposome membrane lipid results in an increase of viscosity. The amount of chemicals enclosed in liposomes, that is, the weight ratio (g/g) of all chemicals (including nonionic iodine compounds and auxiliary additives) in an aqueous solution enclosed in liposomes to liposome membrane lipid is 1 to 8, preferably 3 to 8 and more preferably 5 to 8. When the weight ratio of all chemicals enclosed in liposomes is less than 1, a relatively high content of lipid results in increased viscosity of the preparation, leading to lowered delivery efficiency of chemicals. Unilamellar or several-lamellar liposomes are advantageously superior in enclosure volume and delivery efficiency. On the contrary, when the weight ratio of all chemicals enclosed in liposomes exceeds 8, the liposome structure becomes unstable, inevitably resulting in diffusion or leakage of chemicals beyond the liposome membrane.

Radiographic Contrast Medium

The embodiments of the invention include a liposome-containing preparation obtained by the afore-mentioned manufacturing method of the invention. In one of the preferred embodiments of the liposome-containing preparation, the preparation which includes a nonionic iodine compound as water-soluble chemicals and at least one physiologically acceptable auxiliary additive, is usable as a radiographic contrast medium. The concentration thereof, depending on the objective of imaging, the site, properties of compounds of the contrast medium and the condition of the patient, can optionally be controlled. The optimal dosage of the contrast medium is usually set taking into account the foregoing conditions. The total amount of the iodine compound within and outside the liposomes may be at an extent identical to the conventional dosage. A solution of an excessively high concentration may cause unwanted conditions such as coagulation of liposomes and increased viscosity. The radiographic contrast medium of the invention has an iodine content of 40 to 450 mgI/mL and preferably 70 to 400 mgI/mL at 10 to 300 ml of the expected normal dosage of a preparation solution. The iodine content is preferably 100 to 350 mgI/mL and more preferably 150 to 300 mgI/mL in terms of efficiency of encapsulating contrast medium material within liposomes. An iodine compound and an auxiliary additive are each preferably at a substantially same concentration between inside and outside the lipid membrane.

The overall lipid amount of the radiographic contrast medium is preferably 20 to 100 mg per mL of the contrast medium, and more preferably 20 to 80 mg per mL. The overall lipid means all the kinds of lipids constituting liposomes, such as phospholipid, sterol and glycol. The overall lipid amount may be regarded nearly as the amount of liposomes contained in the contrast medium. Liposomes may be in a variety of forms as long as the overall lipid amount does not simply correspond to the b of the liposomes. In the manufacturing method of the invention, formation of liposomes is efficiently performed and as the lipid amount is increased to a certain amount, the enclosure ratio tends to increase.

The contrast medium of the invention is usable through general dosage or local dosages and preferably, it is generally dosed intravenously as an injection or drip-feed. In cases when local direct dosage to the site incapable of being is not feasible, dosage can be performed by other techniques known in the art, using a catheter or other appropriate drug delivery systems. To lower injection resistance to reduce pain to the examinee and also to avoid crisis of diapedesis, the viscosity of the solution of the composition of the invention is preferably not more than 30 mPa·s at 37° C. (when measured in the Ostwald method), and more preferably not more than 25 mPa·s. The foregoing region causes no problem in practice, as described in Japanese Patent No. 2619037. The osmotic molar concentration of the contrast medium is typically 250 to 500 mosmol/L, and preferably 290 to 350 mosmol/L. A relatively high osmole concentration imposes a burden on the heart and a circulatory system. A solution or suspension which is isotonic to blood is prepared by allowing a contrast medium material to be dissolved or suspended in a medium at a concentration giving an isotonic solution. For example, in cases when an isotonic solution cannot be prepared with only contrast medium compound, due to its low solubility, a nontoxic water-soluble material, such as salts of sodium chloride or saccharides, e.g., mannitol, glucose, saccharose and sorbitol may be added to form an isotonic solution or suspension.

### EFFECT OF THE INVENTION

A liposome-containing preparation allows water-soluble chemicals (preferably nonionic iodine compounds) to be enclosed (or encapsulated) within liposomes as a micro-carrier at a relatively high enclosure ratio of 25-35% and efficient carriage thereof provides high targeting ability.

The liposomes are provided with useful characteristics for use as a radiographic contrast medium. The liposome-containing preparation exhibits superior performance of contrast medium, compared to conventional contrast medium, enabling the use at a lesser amount.

The preparation of the invention is manufactured without using highly toxic chlorinated solvents or other organic solvents and exhibits markedly reduced toxicity and adverse effects, compared to conventional liposome-containing preparations. Accordingly, it lessens the burden on the patient to be dosed.

### EXAMPLES

The embodiments of the invention will be further described based on examples but are by no means limited to these.

### Example 1

A mixture of 340.2 mg of dimyristoylphosphatidylcholine (DMPC), 147.4 mg of cholesterol and 111.5 mg of PEG-phospholipid (SUNBRIGHT DSPE-020CN, lipid modified with polyethylene glycol, product by NIPPON OIL &FATS CO., LTD.) was placed into a stainless steel autoclave. Subsequently, 13 g of liquid carbon dioxide was added thereto and made to the supercritical state by applying pressure at 12 MPa and 33° C. Further thereto, 10 ml of a contrast medium solution (containing iopamidol solution at an iodine content of 300 mg/ml, tromethamol of 1 mg/ml and edetate calcium disodium (EDTA Na₂—Ca) of 0.1 mg/ml) was continuously added using a metering pump with stirring. After completion of the addition, the interior of the autoclave was evacuated to discharge the carbon dioxide, whereby a dispersion of liposomes enclosing the contrast medium solution was obtained.

### Example 2

A mixture of 396.6 mg of distearoylphosphatidylcholine (DSPC), 147.4 mg of cholesterol and 111.5 mg of PEG-phospholipid (SUNBRIGHT DSPE-020CN, product by NIPPON OIL &FATS CO., LTD.) was placed into a stainless steel autoclave. Subsequently, 13 g of liquid carbon dioxide was added thereto and made to the supercritical state by applying pressure at 12 MPa and 65° C. Further thereto, 10 ml of a contrast medium solution (containing iopamidol solution at an iodine content of 300 mg/ml, tromethamol of 1 mg/ml and edetate calcium disodium (EDTA Na₂—Ca) of 0.1 mg/ml) was continuously added using a metering pump with stirring. After completion of the addition, the inside of the autoclave was evacuated to discharge the carbon dioxide, whereby a dispersion of liposomes enclosing the contrast medium solution was obtained.

### Example 3

A mixture of 368.4 mg of dipalmitoylphosphatidylcholine (DPPC), 147.4 mg of cholesterol and 111.5 mg of PEG-phospholipid (SUNBRIGHT DSPE-020CN, lipid modified with polyethylene glycol, product by NIPPON OIL &FATS CO., LTD.) was placed into a stainless steel autoclave. Subsequently, 13 g of liquid carbon dioxide was added thereto and made to the supercritical state by applying pressure at 12 MPa and 50° C. Further thereto, 10 ml of a contrast medium solution (containing iopamidol solution at an iodine content of 300 mg/ml, tromethamol of 1 mg/ml and edetate calcium disodium (EDTA Na₂—Ca) of 0.1 mg/ml) was continuously added using a metering pump with stirring. After completion of the addition, the inside of the autoclave was evacuated to discharge the carbon dioxide, whereby a dispersion of liposomes enclosing the contrast medium solution was obtained. The obtained dispersion was heated to 80° C. and subjected to pressure filtration with 0.8 μm and 0.4 μm polycarbonate filters, using a hydrostatic extruder, produced by Advantech Co. to obtain a dispersion of liposomes containing a contrast medium solution.

### Example 4

A mixture of 368.4 mg of dipalmitoylphosphatidylcholine (DPPC), 147.4 mg of cholesterol and 111.5 mg of PEG-phospholipid (SUNBRIGHT DSPE-020CN, lipid modified with polyethylene glycol, product by NIPPON OIL &FATS CO., LTD.) was placed into a stainless steel autoclave. Subsequently, 13 g of liquid carbon dioxide was added thereto and made to the supercritical state by applying pressure at 12 MPa and 50° C. Further thereto, 10 ml of a contrast medium solution (containing iohexol solution at an iodine content of 150 mg/ml, tromethamol of 1 mg/ml and edetate calcium disodium (EDTA Na₂—Ca) of 0.1 mg/ml) was continuously added using a metering pump with stirring. After completion of the addition, the inside of the autoclave was evacuated to discharge the carbon dioxide, whereby a dispersion of liposomes enclosing the contrast medium solution was obtained. The obtained dispersion was heated to 80° C. and subjected to pressure filtration with 0.8 μm and 0.4 μm polycarbonate filters, produced by Advantech Co., similarly to Example 3 to obtain a dispersion of liposomes containing a contrast medium solution. The dispersion was subjected to ultrafiltration using a ultrafilter (produced by Advantech Co., fractionated molecular weight of 20,000) to obtain a concentrated liposome dispersion.

### Example 5

A mixture of 396.6 mg of distearoylphosphatidylcholine (DSPC), 147.4 mg of cholesterol and 111.5 mg of PEG-phospholipid (SUNBRIGHT DSPE-020CN, product by NIPPON OIL &FATS CO., LTD.) was placed into a stainless steel autoclave. Further thereto, 10 ml of a contrast medium solution (containing iopamidol solution at an iodine content of 300 mg/ml, tromethamol of 1 mg/ml and edetate calcium disodium (EDTA Na₂—Ca) of 0.1 mg/ml) was continuously added using a metering pump with stirring. Subsequently, 13 g of liquid carbon dioxide was added thereto and made to the supercritical state by applying pressure at 12 MPa and 60° C. After completion of the addition of carbon dioxide, the inside of the autoclave was evacuated to discharge the carbon dioxide, whereby a dispersion of liposomes enclosing the contrast medium solution was obtained.

The obtained dispersion was heated to 80° C. and subjected to pressure filtration using 0.8 μm and 0.4 μm polycarbonate filters, produced by Advantech Co., similarly to Example 3 to obtain a dispersion of liposomes containing a contrast medium solution. The dispersion was incubated at 45° C. for 1 hr to obtain an objective liposome dispersion.

### Comparative Example 1

A mixture of 368.4 mg of dipalmitoylphosphatidylcholine (DPPC), 147.4 mg of cholesterol and 111.5 mg of PEG-phospholipid (SUNBRIGHT DSPE-020CN, lipid modified with polyethylene glycol, product by NIPPON OIL &FATS CO., LTD.) was mixed with 10 ml of a mixture of chloroform, ethanol and water (weight ratio; 100:20:0.1) in a messflask. The obtained mixture was heated in a water bath (65° C.) and solvents were evaporated using a rotary evaporator. The residue was dried in vacuo for 2 hr. to form lipid film. Further thereto, 10 ml of a contrast medium solution (containing iopamidol solution at an iodine content of 300 mg/ml) was added and the mixture was stirred by a mixer for 10 min. with heating at 50° C. to obtain a dispersion of liposomes containing the contrast medium solution. The obtained dispersion was subjected to pressure filtration with 0.8 μm and 0.4 μm polycarbonate filters, produced by Advantech Co., similarly to Example 3 to obtain a dispersion of liposomes containing a contrast medium solution. The dispersion was incubated at 45° C. for 1 hr to obtain an objective liposome dispersion.

### Comparative Example 2

A mixture of 340.2 mg of dimyristoylphosphatidylcholine (DMPC), 147.4 mg of cholesterol and 111.5 mg of PEG-phospholipid (SUNBRIGHT DSPE-020CN, lipid modified with polyethylene glycol, product by NIPPON OIL &FATS CO., LTD.) was placed into a stainless steel autoclave. Subsequently, 13 g of liquid carbon dioxide was added thereto and made to the supercritical state by applying pressure at 12 MPa and 70° C. Further thereto, 10 ml of a contrast medium solution (containing iopamidol solution at an iodine content of 300 mg/ml, tromethamol of 1 mg/ml and edetate calcium disodium (EDTA Na₂—Ca) of 0.1 mg/ml) was continuously added using a metering pump with stirring. After completion of the addition, the inside of the autoclave was evacuated to discharge the carbon dioxide, whereby a dispersion of liposomes enclosing the contrast medium solution was obtained.

TABLE 1

| | | | | | Average | |
|---|---|---|---|---|---|---|
| | | Vessel | | | Particle | |
| Example | | Temperature | | Enclosure | Size | Polydisperse |
| No. | Phospholipid | (° C.) | Extruder*² | Ratio (%) | (μm) | Index |
| 1 | DMPC | 33 | | 16 | 1.213 | 0.830 |
| 2 | DSPC | 65 | | 19 | 0.948 | 0.435 |
| 3 | DPPC | 50 | Yes | 24 | 0.274 | 0.051 |
| 4 | DPPC | 50 | Yes | 34 | 0.271 | 0.066 |
| 5 | DSPC | 60 | Yes | 17 | 0.298 | 0.013 |
| Comp. 1*¹ | DPPC | 50 | Yes | 5 | 0.585 | 0.189 |
| Comp. 2 | DMPC | 70 | | 3 | 1.346 | 0.933 |

*¹Bangham method
*²use of a hydrostatic extruder

Evaluation

Thus obtained dispersions containing liposomes enclosing water-soluble chemicals were each measured with respect to the particle size of liposomes and its polydispersity index, using a dynamic light scattering particle size measurement device (Malvern HPPS, produced by Simex Co.) at 25° C. The particle size was represented by an average particle size, based z-average. The polydispersity index indicates the degree of polydispersity. The polydispersity index closer to 0 indicates that the particle size distribution is closer to monodispersity.

What is claimed is:

1. A method of manufacturing a liposome-containing preparation comprising the steps of:
(a) mixing one or more constituents of a liposome membrane, an aqueous solution of a water-soluble chemical and supercritical carbon dioxide at a temperature of 32 to 65° C. in a pressure vessel, and
(b) evacuate the carbon dioxide to form an aqueous dispersion of liposomes enclosing an aqueous solution of a water-soluble chemical,
wherein the constituents include at least one phospholipid exhibiting a transition temperature.

2. The method of 1, wherein step (a) comprises the steps of:
(i) mixing the constituents of a liposome membrane and supercritical carbon dioxide at a temperature of 32 to 65° C. in the pressure vessel to form a suspension, and
(ii) mixing the aqueous solution of a water-soluble chemical with the suspension to obtain a mixture.

3. The method of claim 1, wherein step (a) comprises the steps of:
(i) mixing liquefied carbon dioxide with a suspension of the constituents of a liposome membrane and the aqueous solution of a water-soluble chemical in the pressure vessel to form a mixture, and
(ii) applying a pressure to the mixture at a temperature of 32 to 65° C. with stirring the mixture to bring the liquefied carbon dioxide to supercritical carbon dioxide.

4. The method of claim 1, wherein the method further comprises:
(c) filtering the aqueous dispersion of liposomes under a pressure of 0.01 to 0.8 MPa at a temperature of 50 to 90° C. to obtain the liposomes having an average particle size of 0.05 to 0.8 μm.

5. The method of claim 4, wherein said filtering is conducted using a hydrostatic extruder provided with a membrane filter with pores having a pore size of 0.1 to 1 μm.

6. The method of claim 4, wherein after completion of step (b) or (c), the aqueous dispersion of liposomes is subjected to ultrafiltration.

7. The method of claim 6, wherein after subjected to the ultrafiltration, the liposomes enclose the water-soluble chemical at an enclosure ratio of 25% to 35%.

8. The method of claim 6, wherein after subjected to the ultrafiltration, the aqueous dispersion of liposomes is further subjected to steam sterilization at 115 to 140° C.

9. The method of claim 1, wherein after completion of step (b), the aqueous dispersion of liposomes is subjected to incubation at a temperature of the transition temperature of the phospholipid to the transition temperature plus 10° C. over a period of 0.1 to 3 hr.

10. The method of claim 1, wherein a concentration of the water-soluble chemical contained in a water phase is substantially equal between an interior and exterior of the liposome membrane.

11. The method of claim 3, wherein the suspension is manufactured by mixing the constituents of liposome membrane and the aqueous solution of a water-soluble chemical and is supplied to the pressure vessel.

12. The method of claim 1, wherein the transition temperature is from 22 to 60° C.

13. The method of claim 1, wherein at least 70% of the liposomes is accounted for by vesicles having a lipid membrane formed of two to ten lamellas.

14. The method of claim 1, wherein the constituents of a liposome membrane include a phospholipid, a cationic lipid, a polyethylene glycol-modified lipid and sterols, a molar ratio of (polyethylene glycol-modified lipid)/sterols is within the range of from 100/60 to 100/90 and a molar ratio of phospholipid/sterols is within the range of from 100/5 to 100/25, provided that the phospholipid does not include a polyethylene glycol-modified phospholipid.

15. The method of claim 1, wherein the water-soluble chemical comprises a material used for a radiographic contrast medium.

16. The method of claim 15, wherein the material is a nonionic iodine compound.

17. The method of claim 15, wherein the water-soluble chemical further comprises at least one selected from the group of a buffering agent and a chelating agent.
